(19) United States
(12) Patent Application Publication
(10) Pub. No.: US 2021/0267912 A1
(43) Pub. Date: Sep. 2, 2021

(54) METHYL SULFONYL METHANE-CONTAINING COMPOSITION FOR PREVENTING OR ALLEVIATING OBESITY, FATTY LIVER, AND DIABETES

(71) Applicants: Seung Hoon LEE, Seoul (KR); Tae Kyung LEE, Incheon (KR)

(72) Inventors: Seung Hoon LEE, Seoul (KR); Tae Kyung LEE, Incheon (KR)

(21) Appl. No.: 17/252,676

(22) PCT Filed: Oct. 25, 2018
(86) PCT No.: PCT/KR2018/012675

§ 371 Date: Dec. 15, 2020

(30) Foreign Application Priority Data
Aug. 17, 2018 (KR) 10-2018-0095802

(51) Int. Cl. A61K 31/10; A61K 31/702; A61K 31/519; A61K 31/4415; A61K 33/06; A61K 36/899; A61K 31/375; A61P 3/04; A61P 3/06; A61P 1/16; A61P 3/10; A23L 33/00; A23L 33/15; A23L 33/125; A23L 33/16; A23L 33/105

(52) U.S. Cl. CPC A61K 31/10; A23V 2002/00; A61K 31/519; A61K 31/4415; A61K 33/06; A61K 36/899; A61K 31/375; A61P 3/04; A61P 3/06; A61P 1/16; A61P 3/10; A23L 33/40; A23L 33/15; A23L 33/125; A23L 33/16; A23L 33/105; A61K 31/702

(57) ABSTRACT

The present invention relates to a methyl-sulfonyl methane containing composition for preventing or alleviating obesity, fatty liver and diabetes. An MSM (methyl sulfonyl methane: dimethyl sulfone)-containing composition according to the present invention, which contains 75 to 85 wt. % of MSM, 0.5 to 2 wt. % of vitamin B6 hydrochloride, 0.01 to 1 wt. % of folic acid, 2 to 8 wt. % of galacto-oligosaccharide, 10 to 18 wt. % of rice-fermented magnesium, and 0.5 to 3 wt. % of vitamin C, not only can prevent and treat obesity by reducing the size of fat cells and inhibiting differentiation of mature fat cells, but also lowers triglyceride and the total cholesterol concentration in the liver, thus having a fatty liver preventing effect, and also reduces the concentration of insulin-like growth factor, thus also being effective for diabetes. Therefore, the MSM-containing composition is expected to be useful as a pharmaceutical composition and food composition for preventing and alleviating obesity, fatty liver and diabetes.

### FIELD OF INVENTION

The present invention relates to a composition for preventing or alleviating obesity, fatty liver and diabetes, which contains methylsulfonylmethane.

### BACKGROUND OF INVENTION

As visceral fat-type obesity increases due to changes in dietary habits, development of metabolic syndrome involving fatty liver, abnormal lipid metabolism, diabetes, insulin resistance, high blood pressure, etc. is becoming an ever greater societal problem. These diseases increase the risk of mutual development thereof and are common diseases associated with multiple metabolic changes such as aging, stress, and immune dysfunction.

The main causes of fatty liver are alcoholism, obesity and diabetes mellitus due to overeating, and fatty liver may also occur in other cases including use of medicine with strong toxicity, misuse of antibiotics during pregnancy, abuse of aspirin for alleviating fever of young children who have infectious diseases, etc. Such fatty liver may be easily recovered by regeneration of hepatocytes if physical conditions such as immune status are favorable. If not, fat mass in hepatocytes grows to push important components in the cells including nuclei to one side, which in turn deteriorates functions of hepatocytes. Further, due to accumulated fat in the cells, expanded hepatocytes may press blood vessels and lymphatic glands therebetween, hence causing impaired circulation of blood and lymphatic fluid in the liver. Further, hepatocytes cannot properly receive oxygen and nutrients, thus deteriorating liver function, which in turn causes progression to hepatocirrhosis.

Hyperlipidemia is a condition in which too many fatty components are present in the blood and accumulated on a blood vessel wall, causing inflammation and therefore cardiovascular disease. Hyperlipidemia is classified into hypertriglyceridemia, hyperemia and hypercholesterolemia and, recently, may also include so-called dyslipidemia that includes increase in triglycerides, total cholesterol and LDL-cholesterol, or reduced HDL-cholesterol in blood. This disease has no specific symptom but causes arteriosclerosis, which may lead to coronary heart disease, cerebrovascular disease, and peripheral vascular occlusion. In addition, excessive fat accumulation may also affect the liver and therefore is closely related to fatty liver development.

Obesity is not just high weight but a state of excessive accumulation of body fat. This means that, even if a person looks normal but has a high body fat percentage, this state may refer to obesity. In general, obesity is determined by body mass index (BMI). More particularly, a BMI of 23 to 24.9 is classified as overweight, while a BMI of 25 to 29.9 is classified as mild obesity. Further, if BMI is 30 to 34.9, this is determined as moderate obesity, while being extremely obese when BMI is 35 or more. Obesity is caused by a combination of multiple factors rather than a single cause, for example, bad dietary habits including westernized dietary habits, decreased performance, emotional factors, genetic factors, etc. Such developed obesity may therefore increase the risk of developing chronic diseases including hyperlipidemia, diabetes, high blood pressure, etc.

Obesity refers to a state in which body fat is excessively accumulated and corresponds to a body fat percentage of 25% or more in men and 30% or more in women, or a body mass index (BMI) of 30 kg/m² or more. As a cause of obesity, environmental factors such as excessive caloric intake and lack of exercise due to lifestyle changes have emerged. Meanwhile, diabetes associated with obesity is classified into insulin dependent diabetes mellitus (type I diabetes mellitus), insulin independent diabetes mellitus (type II diabetes mellitus), and malnutrition diabetes mellitus (MRDM). Herein, Type II diabetes accounts for more than 90% of Korean diabetes patients, is a metabolic disease characterized by hyperglycemia and is reported to be caused by decreased insulin secretion of pancreatic beta cells or increased insulin resistance in peripheral tissues due to genetic, metabolic and environmental factors. Diabetes is closely related to obesity and prevalence mechanisms. In this regard, when body fat increases with obesity, insulin sensitivity tends to decrease. Further, in case of patients with type II diabetes, obesity is closely correlated with insulin resistance and, therefore, it is known that the greater the obesity, the greater the insulin resistance.

Current medicaments to treat obesity may be generally divided into drugs acting on the central nervous system to affect appetite and drugs acting on the gastrointestinal tract to inhibit absorption. Drugs acting on the central nervous system may include, for example: drugs to inhibit the serotonin (5HT) nervous system according to respective mechanisms such as fenfluramine and dexfenfluramine; drugs acting though the noradrenaline nervous system such as ephedrine and caffeine; and drugs simultaneously acting on both serotonin and noradrenaline nervous systems to inhibit obesity such as sibutramine, which are recently and commercially available. Further, drugs acting on the gastrointestinal tract may include orlistat recently approved as an anti-obesity agent, which is a representative medicine to inhibit lipase produced in the pancreas so as to reduce fat absorption. However, among conventionally used anti-obesity agents, fenfluramine has recently been prohibited due to side effects such as primary pulmonary hypertension and heart valve lesions. Further, sibutramine has side effects of increasing blood pressure, and orlistat is known to have side effects such as digestive problems. In addition, other chemical synthetic drugs also caused problems such as blood pressure reduction or lactic acidosis and, therefore, could not be used in patients with specific diseases such as heart failure and kidney disease.

In other words, a novel method for prevention or treatment of obesity and diabetes closely associated with the same while reducing side effects of existing medicaments is needed. In recent years, research to find a solution from natural materials is being actively performed. That is, there is a requirement for a treatment method with anti-obesity and anti-diabetic functions through effects of body weight gain inhibition, reduced fat accumulation, fat cell size reduction, fatty liver improvement, blood lipid alleviation, blood sugar reduction and reduced insulin resistance, using natural materials. The present invention has been proposed in view of such points.

Diabetes is a disease in which glucose present in the blood is excreted through urine, and is a metabolic disorder of multiple etiologies characterized by chronic hyperglycemia due to a lack of insulin secretion or a lack of insulin action. Diabetes may be roughly divided into type I diabetes (insulin-dependent) and type II diabetes (insulin-independent). The etiology of type II diabetes is known to be a combination of insulin secretion disorder and insulin resistance.

More than 95% of diabetes mellitus is type II diabetes, which is rapidly increasing due to changes in the living environment including westernized dietary habits, increased stress and aging population, according to rapid economic development.

Diabetes, known as a representative adult metabolic disease, is also progressing to digestive diabetes, which in turn causes serious concerns. About 5% of the world's population suffers from diabetes and the prevalence of diabetes mellitus in Korea is between 5 and 10%. As of 2014, 20.7 deaths per 100,000 population among the major causes of death were cancer, heart disease, cerebrovascular disease, ischemic heart disease and diabetes in sequential order, resulting in enormous human and economic losses (National Statistical Office, 2015).

Insulin secretion disorder refers to a situation in which an appropriate amount of insulin is not secreted from beta cells of the pancreas depending on the blood glucose level. This is due to the quantitative decrease in insulin secreting beta cells or impaired functional distribution of beta cells.

Insulin resistance refers to a situation in which secreted insulin has reached the target organ via the bloodstream, however, insulin action and sensitivity are reduced in the target cell. In general, insulin resistance is thought to be a signaling disorder in which intracellular signals generated after binding insulin to a cell membrane insulin receptor has reduced signal intensity due to some causes. Insulin secretion disorder and insulin resistance may act in combination with each other to make diabetes worse. In other words, if insulin resistance is present, a larger amount of insulin must be secreted to overcome the same. On the contrary, hyperglycemia caused by insufficient insulin secretion may further worsen insulin resistance.

Current treatment methods for insulin-independent diabetes include, for example, meal therapy, exercise therapy and pharmacotherapy. Pharmacotherapy is applied to clinical treatment using various drugs, for example: sulfonylurea-based drugs to increase insulin secretion; peroxisome proliferator activated receptor gamma (PPAR-γ)-enhancing drugs to improve insulin activity; α-glucosidase inhibitor-based drugs (e.g. acarbose) to inhibit digestion and absorption of carbohydrates and to prevent elevation of postprandial blood sugar level; biguanide-based drugs (e.g. metformin) to promote glucose uptake into cells; insulin with different durations, etc.

However, such therapeutic agents have problems of low efficacy or different side effects such as liver dysfunction, hypoglycemia, lactic acidosis and the like. Therefore, it is necessary to develop a safe diabetes therapeutic agent available for long-term use while having reduced side effects. In response to such requirements, studies are being actively conducted to search for diabetes treatment drugs from natural materials. In 1990, the World Health Organization (WHO) strongly recommended the use of natural products that are effective in diabetes and have reduced side effects (Grover J K et al., J. Ethnopharmacol., 73, pp 461-470, 2000).

However, although strict blood glucose control is known to prevent or delay the development of diabetic complications, drugs effective in treating type II diabetes are insufficient for strict blood glucose control or for preventing complications of diabetes. Therefore, a novel therapeutic agent is required.

Meanwhile, sulfur is an oxygen group element, that is, a chalcogen (element symbol: S, atomic number: 16, atomic weight: 32.06, melting point: 112.8° C. (α sulfur), boiling point: 444.7° C., specific gravity: 2.07 (α-sulfur)), which is produced in a free state as natural sulfur on the earth, but is also broadly present as a compound. Sulfur is the fourth most abundant mineral in the body after calcium, phosphorus and potassium, is found in every cell in the body, is a component of major amino acids such as cystine, cysteine and methionine, and is essential for the life of cells. Further, sulfur is a component of vitamins such as thiamin and biotin and is also present in saliva, bile and hormone insulin.

Sulfur accounts for 0.15% of the human body weight and also for 10% of the mineral content in the body. As such, sulfur is one of the most important substances in metabolic activities such as breathing and assimilation of living things. Since ancient times, sulfur is known to be effective in conserving human vigor. Regularly prepared sulfur is the best remedy to treat a lack of vigor and has been used as a medicine for gynecological pediatric diseases, including various ulcers, inflammations and colds. According to Huh Jun's DonguiBogam (“The Principles and Practice of Eastern Medicine”), “Sulfur has a lot of heat and strong toxicity, but may remove chill in the body to help a lack of vigor, pathologic accumulation (a disease that mass is generated in the stomach due to long term indigestion in Chinese medicine) and poor energy (bad energy that brings illness to the body, such as exterior fever and chill, in Chinese medicine). Further, sulfur has detoxification effects.

Methylsulfonylmethane (MSM: dimethyl sulfone) is a dietary form of sulfur that is nontoxic and odorless, and is found in the ocean, the atmosphere, plant tissues, animal tissues and human bodies. This compound is extracted from California Bristlecone pines and is a natural product made from lignin which is a natural organic sulfur compound as a biomaterial. As a result of animal toxicity test, the half lethal dose of MSM is more than 20 g per 1 kg of body weight, which is equal to the half lethal dose of water. Therefore, MSM has safety substantially similar to water and, even if it is taken for a long term, is safe. With regard to efficacy of MSM, it is known that MSM is effective in alleviating pain, skin redness and itching of knee osteoarthritis. Further, according to literature [MSM Miracle. The Official website of the MSM Medical Information Foundation. 2005-2006], it is reported that MSM is effective in treating inflammation caused by autoimmune diseases.

Vitamin B6 hydrochloride is a water-soluble vitamin not stored by the body and is excreted in urine. Vitamin B6 exists in three forms: pyridoxal, pyridoxine and pyridoxamine. Vitamin B6 is involved in energy metabolism, especially protein metabolism, and is significant for circulatory, nervous and immune systems. Folic acid is a type of vitamin, also called vitamin B9 or vitamin M. Because of importance for development of nerves and blood vessels in the fetus, this is recommended for pregnant women before and during pregnancy and is abundant in fruits.

Therefore, during continuous research to develop functional foods including MSM as an active ingredient, the present inventors have found that a composition containing vitamin B6 hydrochloride and folic acid as well as MSM may reduce a size of adipocytes and inhibit differentiation of mature adipocytes so as to prevent and treat obesity, may decrease triglyceride and total cholesterol levels in the liver to exhibit fatty liver preventing effects, in addition, may reduce a concentration of insulin-like growth factor so as to exhibit effects on diabetes, thereby completing the present invention.

### SUMMARY OF INVENTION

### Technical Problem to be Solved

A technical problem to be solved in the present invention, that is, an object of the present invention, is to provide a pharmaceutical and food composition for preventing or alleviating obesity.

Another object of the present invention is to provide a pharmaceutical and food composition for preventing or improving fatty liver.

A further object of the present invention is to provide a pharmaceutical and food composition for preventing or alleviating diabetes.

### Technical Solution

In order to solve the above technical problems, that is, in order to accomplish the above objects, the present invention may provide a pharmaceutical and food composition for preventing or alleviating obesity, which includes 75 to 85% by weight (“wt. %”) of methylsulfonylmethane (MSM: dimethyl sulfone), 0.5 to 2 wt. % of vitamin B6 hydrochloride, 0.01 to 1 wt. % of folic acid, 2 to 8 wt. % of galactooligosaccharide, 10 to 18 wt. % of rice fermented magnesium, and 0.5 to 3 wt. % of vitamin C.

In order to accomplish the above objects, the present invention may provide a pharmaceutical and food composition for preventing or improving fatty liver, which includes 75 to 85 wt. % of methylsulfonylmethane (MSM: dimethyl sulfone), 0.5 to 2 wt. % of vitamin B6 hydrochloride, 0.01 to 1 wt. % of folic acid, 2 to 8 wt. % of galactooligosaccharide, 10 to 18 wt. % of rice fermented magnesium and 0.5 to 3 wt. % of vitamin C.

In order to accomplish the above objects, the present invention may provide a pharmaceutical and food composition for preventing or alleviating diabetes, which includes 75 to 85 wt. % of methylsulfonylmethane (MSM: dimethyl sulfone), 0.5 to 2 wt. % of vitamin B6 hydrochloride, 0.01 to 1 wt. % of folic acid, 2 to 8 wt. % of galactooligosaccharide, 10 to 18 wt. % of rice fermented magnesium and 0.5 to 3 wt. % of vitamin C.

The composition of the present invention may include to 85 wt. % of MSM based on a total weight of the composition. At this time, when a content of MSM is less than 75 wt. %, effects of preventing obesity, fatty liver and diabetes, which are objective effects of the present invention, cannot be obtained. On the other hand, when a content of MSM is more than 85 wt. %, the above effects are not proportional to an increase in content, that is, the inventive composition may be inefficient. In addition, there is a problem of not ensuring formulation stability.

The pharmaceutical composition may have any one formulation selected from the group consisting of tablets, pills, powders, granules, capsules, suspensions, liquid solutions, emulsions, syrups, sterile aqueous solutions, non-aqueous solvents, lyophilizers and suppositories, which may be a variety of oral or parenteral formulations. When formulated, diluents or excipients such as fillers, extenders, binders, wetting agents, disintegrating agents and surfactants are usually used to prepare the formulation. Solid form preparations for oral administration may include tablets, pills, powders, granules, capsules and the like, and such solid form preparations may be prepared by mixing at least one compound with at least one excipient, for example, starch, calcium carbonate, sucrose or lactose, gelatin, etc. Further, in addition to simple excipients, lubricants such as magnesium stearate, talc, etc. are also used. Liquid preparations for oral administration may include suspensions, liquid solutions, emulsions, syrups, etc. Other than simple and commonly used diluents such as water and liquid paraffin, diverse excipients, for example, wetting agents, sweeteners, fragrances, preservatives, etc. may also be included. Formulations for parenteral administration may include sterile aqueous solutions, non-aqueous solvents, suspensions, emulsions, lyophilized preparations and suppositories. As the non-aqueous solvent and the suspension solvent, propylene glycol, polyethylene glycol, vegetable oils such as olive oil, injectable esters such as ethyl oleate, etc. may be used. As a basic material of the suppository, Witepsol, macrogol, tween 61, cacao butter, laurin butter, glycerogelatin, etc. may be used.

The composition of the present invention may be administered in a pharmaceutically effective amount. As used herein, the term “pharmaceutically effective amount” means an amount sufficient to treat a disease at a reasonable benefit/risk ratio applicable to medical treatment, and an effective dose level may be determined in consideration of individual type and severity, age, gender, type of disease, activity of drug, sensitivity to drug, time of administration, route of administration, release rate, duration of treatment, factors including concurrently used drugs, other factors well known in the field of medicine. The composition of the present invention may be administered as a therapeutic agent alone or in combination with other therapeutic agents, and may be administered sequentially or simultaneously with conventional therapeutic agents. Further, single or multiple administrations may be possible. Taking all of the above factors into consideration, administering an amount that can afford maximum effects in a minimum amount without side effects is important, and such amount may be easily determined by those skilled in the art. Preferred dosage of the composition of the present invention may depend on condition and body weight of a patient, extent of disease, drug type, administration route and duration of administration. In fact, a suitable total daily dose may be determined by a practitioner with suitable medical experience. Generally, an amount of 0.001 to 1000 mg/kg, preferably 0.05 to 200 mg/kg, and more preferably 0.1 to 100 mg/kg may be administered once or several times per day. The composition is not particularly limited as long as this is applied to a subject for alcohol decomposition purposes, and is applicable to any subject. For example, the composition may be applied to any individual such as non-human animals including, for example, monkeys, dogs, cats, rabbits, marmots, rats, mice, cattle, sheep, pigs, goats, etc., as well as humans, birds and fishes. A mode of administration may include any conventional method used in the art without limitation. For example, the composition may be administered by oral, rectal or intravenous, intramuscular, subcutaneous, intrauterine or cerebrovascular injection.

As one specific embodiment, the present invention provides a functional food composition for prevention of obesity, fatty liver and diabetes, which contains MSM as an active ingredient.

When using the composition of the present invention in food, the composition may be added as is, or used with other health foods, health functional foods or health functional food ingredients, and may be suitably used in accordance with any conventional method. A mixing amount of the active ingredient may be appropriately determined depending on intended use. In general, the composition of the present invention may be added in an amount of preferably 15 parts by weight (“wt. parts”) or less, more preferably 10 wt. parts or less with respect to the raw material in the manufacture of food or beverage. However, in the case of long-term intake for purposes of health control and hygiene, the amount may be below the above range. Indeed, since no problem is present in terms of stability, the active ingredient may also be used in an amount greater than the above range.

Types of foods possibly including the composition of the present invention are not particularly limited, and specific examples may include meat, sausage, bread, chocolate, candy, snacks, confectionery, pizza, ramen, other noodles, gums and dairy products including ice cream, various soups, beverages, tea, drinks, alcoholic beverages and vitamin complexes, etc., may include all foods within common sense, and may further include foods used as feed for animals. In addition, when the food composition of the present invention is used in the form of a beverage, additional ingredients such as various sweetening agents, flavoring agents or natural carbohydrates, etc. may be contained as in general beverages. Natural carbohydrate may be monosaccharides such as glucose and fructose, disaccharides such as maltose and sucrose, polysaccharides such as dextrin and cyclodextrin, and sugar alcohols such as xylitol, sorbitol, erythritol, etc. A fraction of the natural carbohydrate is not limited thereto but is preferably about 0.01 to 0.04 g, more preferably 0.02 to 0.03 g with respect to 100 ml of the composition of the present invention. The sweetener may be a natural sweetener such as thaumatin, stevia extract, etc., and a synthetic sweetener such as saccharin, aspartame, etc. In addition to the above, the food composition of the present invention may include various nutrients, vitamins, electrolytes, flavors, coloring agents, pectic acid and salts thereof, alginic acid and salts thereof, organic acids, protective colloid thickeners, pH adjusting agents, stabilizers, preservatives, glycerin, alcohols, carbonation agents used in carbonated beverages, and the like. Others such as pulp for production of natural fruit juices, fruit juice drinks and vegetable drinks may be included.

As used herein, the term “administration” means introducing the pharmaceutical composition of the present invention to an individual in any suitable manner, and the route of administration may include various oral or parenteral routes as long as the composition can reach the target tissue via the route. The treatment method of the present invention may include administering a pharmaceutical composition comprising MSM in a pharmaceutically effective amount. A suitable total daily dose may be determined by a practitioner within a range according to the correct medical judgment, and is generally determined in an amount of 0.001 to 1000 mg/kg, preferably 0.05 to 200 mg/kg, more preferably 0.1 to 100 mg/kg, which may be administered once to several times daily.

However, for the purposes of the present invention, a therapeutically effective amount for a particular patient is preferably and differently applied on the basis of various factors, for example: type and extent of reaction to be achieved; optionally whether or not other agents are used; specific compositions; age, body weight, general state, gender and diet of a patient; administration time, administration route and secretion rate of composition; duration of treatment; and other drugs used along with or concurrent with the specific compositions, as well as other similar factors well known in the field of medicine.

### Effect of Invention

The MSM-containing composition according to the present invention can prevent and treat obesity by reducing a size of adipocytes and inhibiting differentiation of mature adipocytes, can reduce triglycerides and total cholesterol levels in the liver to exhibit fatty liver prevention effects, and is also effective on diabetes by decreasing a concentration of insulin-like growth factors. Therefore, it is expected that the inventive composition may be usefully used as a pharmaceutical and food composition for preventing and alleviating obesity, fatty liver and diabetes.

### BRIEF DESCRIPTION OF DRAWINGS

The following drawings attached to this specification are provided to illustrate exemplary embodiments of the present invention, and will serve to further illustrate the technical spirit of the present invention together with the above-described contents of the present invention. Therefore, it should not be construed that the present invention is limited to the matters described in the drawings.

FIG. 1 is graphs illustrating effects of the MSM-containing composition according to the present invention on change of adipose tissues (epididymal cells) as a result of experiments for different groups, which are divided into: normal diet group (NCD); high fat diet group (HFD); high fat diet and MSM-containing composition intake group (HFD+MSM (0.0088%)); and high fat diet and MSM-containing composition intake group (HFD+MSM (0.017%)).

FIG. 2 is graphs illustrating effects of the MSM-containing composition according to the present invention on change of liver tissues as a result of experiments for different groups, which are divided into: normal diet group (NCD); high fat diet group (HFD); high fat diet and MSM-containing composition intake group (HFD+MSM (0.0088%)); and high fat diet and MSM-containing composition intake group (HFD+MSM (0.017%)).

FIG. 3 is micrographs showing liver tissue morphology in a high fat diet obese mouse model by the MSM-containing composition.

FIG. 4 is micrographs showing liver tissue morphology in a high fat diet obese mouse model by the MSM-containing composition.

FIG. 5 is micrographs showing 3T3-L1 cell differentiation inhibitory effects in a high fat diet obese mouse model by the MSM-containing composition.

FIG. 6 is graphs illustrating measured results of change in liver triglyceride content in obese mice by the MSM-containing composition.

FIG. 7 is graphs illustrating measured results of change in total liver cholesterol content in obese mice by the MSM-containing composition.

FIG. 8 illustrates a result of in vitro lipolysis experiment using milk coffee with the MSM-containing composition.

FIG. 9 illustrates a result of in vitro lipolysis experiment using Korean chicken soup (“Samgyetang”) with the MSM-containing composition.

FIG. 10 is graphs showing IGF-1 secretion ability of the MSM-containing composition.

### DETAILED DESCRIPTION OF PREFERRED EMBODIMENTS OF INVENTION

Hereinafter, preferred examples and the like are provided to facilitate understanding of the present invention. However, the following examples are provided only to more easily illustrate the present invention, and the contents of the present invention are not limited by the same.

### [Example 1] Preparation of Composition Containing methylsulfonylmethane

A composition containing methylsulfonylmethane (“MSM-containing composition”) having a constitutional composition as shown in Table 1 below was prepared.

TABLE 1

| No. | Component | Content (%) |
|---|---|---|
| 1 | MSM | 80.000 |
| 2 | Vit. B6 | 1.100 |
| 3 | Folic Acid | 0.050 |
| 4 | Galactooligosaccharide | 4.000 |
| 5 | Rice fermented magnesium | 13.399 |
| 6 | Vit. C | 1.450 |
| 7 | Vit. B12 mixed powder | 0.001 |
| Total | | 100 |

### <Experimental Example 1> Inhibitory Effects on Body Weight Gain in High Fat Diet Obese Mouse Model by the Composition According to the Present Invention

As experimental animals, C57BL/6 series 8-week-old male mice were adapted with solid feed for 1 week, and were divided into 3 groups with an average body weight of 25 g according to randomized block design and bred for 8 weeks.

The experimental groups were divided into: a normal diet group (NCD); a high fat diet group (HFD); a group having a high fat diet and the MSM-containing composition obtained in Example 1 (HFD+MSM (0.0088%)); a group having a high fat diet and the MSM-containing composition obtained in Example 1 (HFD+MSM (0.017%)), followed by implementing experiments. The normal diet group was fed a general diet wherein fat was 10% of total calories, while the high fat diet group was fed a diet wherein fat was 60% of total calories. Further, with regard to the groups having ingested the high fat diets together with the MSM-containing compositions, the diets fed to these groups were prepared by adding 0.0088% and 0.017% MSM-containing compositions, respectively, to the high fat diets. During the breeding period, water and feed were freely afforded. A temperature in the breeding space was maintained at 22±1° C. while illumination was controlled on 12-hour cycle (08:00 to 20:00). All animal experiments were performed while complying with Animal Experimentation Ethic Code under approval of the Science and Technology Institutional Animal Care and Use Committee.

The results obtained in the experiments were expressed as the average±standard error for each experimental group, and statistical analysis of the mean difference between the two groups was analyzed by Student's t test, followed by verifying significance of the result at P<0.05 level.

Dietary intake and body weight of the test animals were measured once a week. The body weight gain rate of each experimental group was measured at a predetermined time and a predetermined interval of 1 week during the experimental period, and the food efficiency ratio (FER) was calculated by dividing total body weight gain by dietary intake during the experimental period, which was defined as a duration from the feeding date of the experiment diet to the sacrifice date.

Effects of the MSM-containing composition according to the present invention on dietary intake and body weight are shown in Table 2 for individual groups.

TABLE 2

| | | | Food | |
|---|---|---|---|---|
| | Total body | Total | efficiency | |
| Test | weight gain | dietary | ratio | |
| group | (g) | intake (g) | (FER) (%) | p-value |
| NCD | 5.3 | 42.13 | 12.5 ± 1.7 | — |
| HFD | 21.3 | 31.30 | 68.1 ± 3.9 | 0.000 |
| HFD + | 11.7 | 31.32 | 37.9 ± 7.8 | 0.031 |
| MSM | | | | |
| (0.008%) | | | | |
| HFD + | 11.6 | 31.00 | 38.9 ± 4.0 | 0.003 |
| MSM | | | | |
| (0.018%) | | | | |

As shown in Table 2, FER of the high fat diet group was about 5.4 times higher than the normal diet group, but it was found that FER of the high fat diet+MSM-containing composition intake group was decreased by about 1.8 times, as compared to the normal diet group. On the other hand, as compared to the normal diet group, the body weight was rapidly increased in the high fat diet group while the body weight gain in the high fat diet+MSM-containing composition intake group was significantly reduced. Therefore, it could be confirmed that intake of the MSM-containing composition significantly inhibits body weight gain caused by the high fat diet.

### <Experimental Example 2-1> Fat Accumulation Inhibitory Effects of MSM-Containing Composition in High Fat Diet Obese Mouse Model

Breeding, diet and statistical treatment of the experimental animals were performed in the same manner as in Experimental Example 1. In order to confirm fat accumulation inhibitory effects of the MSM-containing composition according to the present invention, the fat and liver tissues of the test animals were separated and weighed.

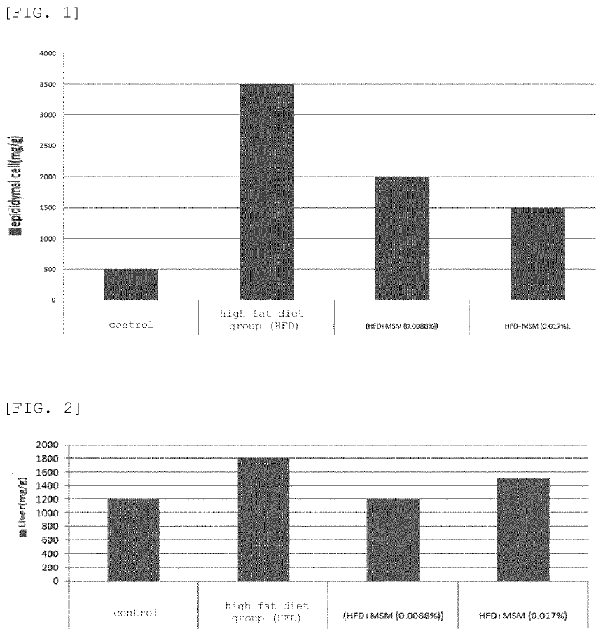

FIG. 1 is graphs illustrating effects of the MSM-containing composition according to the present invention on change of adipose tissues (epididymal cells) as a result of experiments for different groups, which are divided into: normal diet group (NCD); high fat diet group (HFD); high fat diet and MSM-containing composition intake group (HFD+MSM (0.0088%)); and high fat diet and MSM-containing composition intake group (HFD+MSM (0.017%)).

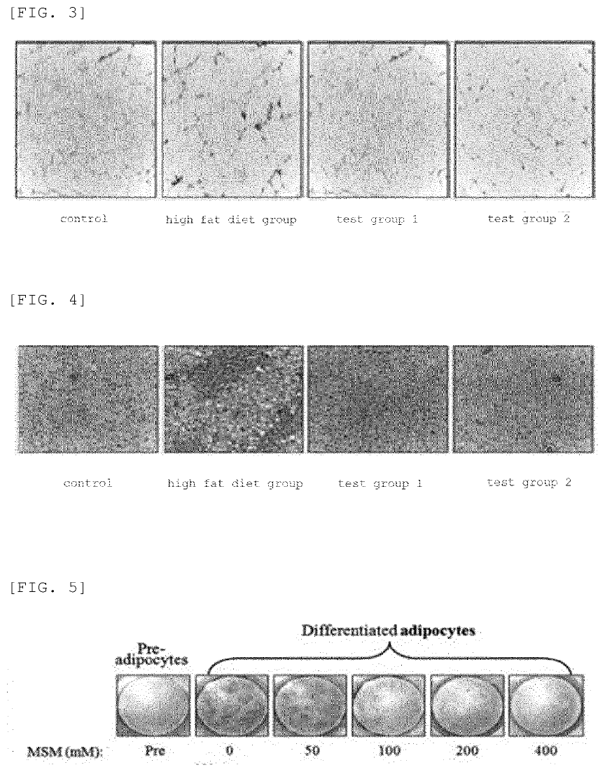

FIG. 2 is graphs illustrating effects of the MSM-containing composition according to the present invention on change of liver tissues as a result of experiments for different groups, which are divided into: normal diet group (NCD); high fat diet group (HFD); high fat diet and MSM-containing composition intake group (HFD+MSM (0.0088%)); and high fat diet and MSM-containing composition intake group (HFD+MSM (0.017%)).

As shown in FIGS. 1 and 2, the weight of adipose tissues in a higher level was measured in the high-fat diet group compared to the normal diet group. On the other hand, it could be seen that that adipose tissues were reduced in the group having the high-fat diet together with the MSM-containing composition.

### <Experimental Example 2-2> Effects of Adipose Cell Size Reduction and Fatty Liver Improvement in High Fat Diet Obese Mouse Model

Breeding, diet and statistical treatment of experimental animals were performed in the same manner as in Experimental Example 1. For morphological observation of the experimental animals, liver tissues were extracted after experiment, and then fixed in a 4% paraformaldehyde solution after water removal. The fixed tissues were washed with running water, followed by dehydration in ethanol in sequential order of increased concentration. Then, the treated tissues were subjected to infiltration and embedding in paraffin, followed by preparing 4 μm tissue sections, staining the same with hematoxylin and eosin. Thereafter, the stained tissues were observed under an optical microscope.

As compared to the normal diet group, the size of adipose cells in the high fat diet group was considerably increased, whereas the high fat diet and MSM-containing composition intake group showed significantly reduced size of adipose cells. Therefore, it could be confirmed that the composition of the present invention is useful for treatment and prevention of obesity caused by high fat diet.

FIG. 3 is micrographs showing liver tissue morphology in a high fat diet obese mouse model by the MSM-containing composition. It was observed in FIG. 3 that fat was accumulated throughout the high fat diet group compared to the normal diet group, while fat accumulation in the high fat diet and MSM-containing composition intake group was significantly reduced to be closer to the normal diet group. Accordingly, it could be assessed that the MSM-containing composition has efficacy in improving fatty liver.

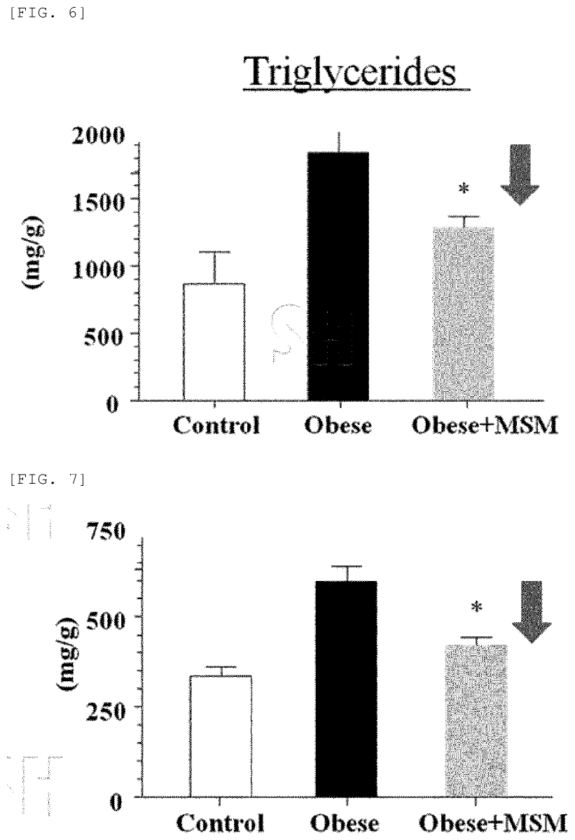

FIG. 4 is micrographs showing liver tissue morphology in a high fat diet obese mouse model by the MSM-containing composition. It was observed in FIG. 4 that fat was accumulated throughout the high fat diet group compared to the normal diet group, while fat accumulation in the high fat diet and MSM-containing composition intake group was significantly reduced to be closer to the normal diet group. Accordingly, it could be assessed that the MSM-containing composition has efficacy in improving fatty liver.

### <Experimental Example 3> Mature Adipocyte Differentiation Inhibitory Effects in High Fat Diet Obese Mouse Model

(1) 3T3-L1 Cell Culture and Differentiation

Mouse-derived 3T3-L1 cells were obtained from the American Type Culture Collection (ATCC, CL-173, Manassas, Va., USA) and used. After seeding 3T3-L1 pre-adipocytes at 1×10⁶ cells/well in 1000, 24-well and 96-well plates, respectively, depending upon purposes of the experiment, the cells were incubated in a high concentration of glucose DMEM (89%) containing BS(10%) and P/S(1%) to reach 100% confluence. 2 days later, the pre-adipocytes were subjected to induction of differentiation using an adipocyte differentiation-inducing substance (1 μg/mL insulin, 1 μM DEX, 0.5 mM IBMX) as well as DMEM containing FBS (10%) and P/S (10%). During adipocyte differentiation (day 0), samples were treated with DMEM at 25, 50 and 100 μg/mL, while a positive control was treated with 5 mM of NAC as an antioxidant, and then used for comparison.

In order to determine 3T3-L1 cell differentiation inhibitory efficacy, an amount of triglycerides generated in 3T3-L1 adipocytes was measured by Oil Red 0 staining to stain only triglycerides in red. A result of the measurement is shown in FIG. 5.

FIG. 5 is micrographs showing 3T3-L1 cell differentiation inhibitory effects in a high fat diet obese mouse model by the MSM-containing composition. As shown in FIG. 5, it could be confirmed that 3T3-L1 cells were not differentiated owing to administration of the MSM-containing composition according to the present invention.

### <Experimental Example 4> Blood Lipid Improvement Effects in High Fat Diet Obese Mouse Model

Breeding, diet and statistical treatment of the experimental animals were performed in the same manner as in Experimental Example 1. In order to confirm effects of improving blood lipids by the MSM-containing composition of the present invention, all animals were subjected to fasting for at least 12 hours after the end of experiment, followed by sacrificing and collecting blood. The collected blood was left at room temperature for 1 hour and then centrifuged at 3000 rpm to obtain serum, which in turn was analyzed using an automatic biochemical analyzer (BS-380, Mindray) to assess intrahepatic triglycerides and total cholesterol.

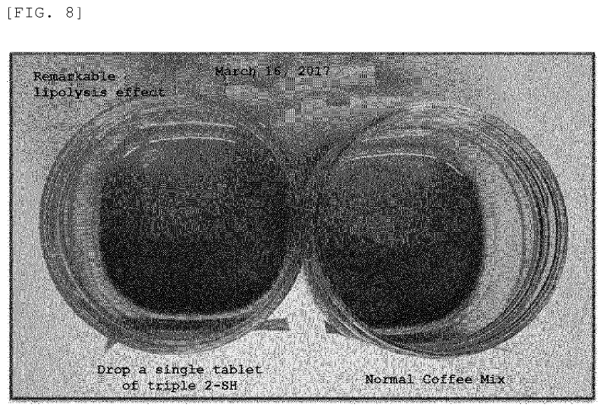

FIG. 6 is graphs illustrating measured results of change in liver triglyceride content in obese mice by the MSM-containing composition.

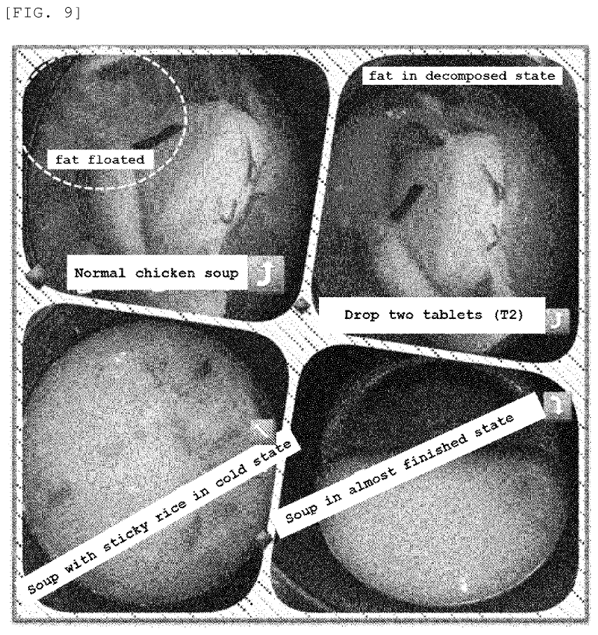

FIG. 7 is graphs illustrating measured results of change in total liver cholesterol content in obese mice by the MSM-containing composition.

As shown in the figures, it could be confirmed that the triglyceride and total cholesterol contents have returned to substantially normal levels as a result of administration of the MSM-containing composition for 3 weeks to the obese mice.

### <Experimental Example 5> Measurement of In Vitro Lipolysis by MSM-Containing Composition

Lipolysis experiments were implemented using normal milk coffee and Korean chicken soup (“Samgyetang”). The milk coffee was added to the MSM-containing composition of the present invention and then dissolved, followed by visual observation of lipolysis.

FIG. 8 illustrates a result of in vitro lipolysis experiment using milk coffee with the MSM-containing composition.

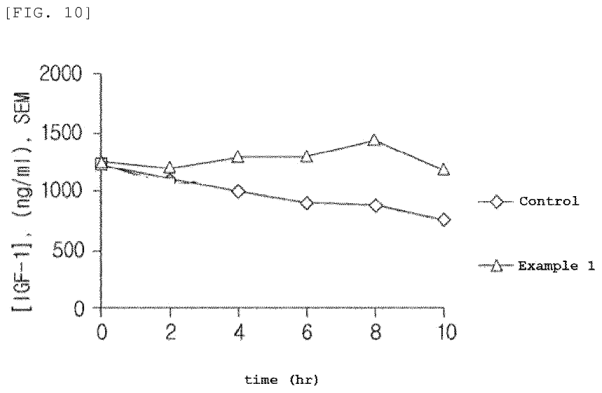

FIG. 9 illustrates a result of in vitro lipolysis experiment using Korean chicken soup (“Samgyetang”) with the MSM-containing composition.

As shown in the figures, it could be visually confirmed that fats were decomposed in the milk coffee and samgyetang to which the MSM-containing composition according to present invention was added, respectively.

### <Experimental Example 6> Determination of Insulin-Like Growth Factor Secretion Ability of MSM-Containing Composition

In the present experimental example, insulin-like growth factor (IGF-1) secretion ability of the MSM-containing composition was tested.

Analyzers used herein were ELISA readers (LAB SYSTEM, USA) and ELISA kits (rat IGF-1, catalog #DSL 10-2900, Diagnostic Systems Laboratories, USA), while a data analysis and statistical system used herein was Prism (ver. 2.01, GraphPad Software Inc., USA).

Sprague Dawley rats were used as test animals. The test animals were 3 weeks of age for the long-term administration test and 9 weeks of age for the IGF-1 evaluation test. The test animals were male and 30 animals were assigned for each test (10 for controls, 10 for examples, 10 for comparative examples). Breeding conditions of the test animals were set to: temperature 22±3 (19 to 25) ° C.; humidity 30 to 70%; photoperiod 12 hours (light cycle: 08:00 to 20:00).

For IGF-1 evaluation test, all animals were used after fasting for 24 hours in order to control a basic amount of growth hormone in the blood or for concurrency. For the test group, a daily dose (1,500 mg/day based on a 60 kg human) was orally administered. The control group was supplied with the same volume of drinking water as the test group. Tail blood was collected at 0 hours and, after oral administration, 0.1 ml or less of blood was collected at 2 hour intervals up to 10 hours, followed by serum separation to prepare a measurement sample. IGF-I measurement was implemented using a kit product by means of ELISA.

Since growth hormone secretion mostly occurs by instantaneous release called pulse mode, it is very difficult to measure a change in growth hormone secretion in response to physiological stimulation. Indeed, although the hormone flows through the bloodstream for only a few minutes, this is enough for the hormone to enter the liver and stimulate the same so as to be converted into growth factors in the liver. IGF-1 secretion is used to measure an amount of growth hormone secretion, wherein IGF-1 has more diverse activities than the growth hormone itself and directly acts on most biological activities. Therefore, in the present test, an amount of blood IGF-1 secretion as a secondary signal of growth hormone was measured, and IGF-1 remained stable in blood after stimulation and substantially showed effects of growth hormone.

FIG. 10 is graphs showing IGF-1 secretion ability of the MSM-containing composition.

As shown in the figure, the amount of IGF-1 secretion in the control group was continuously decreased, whereas the MSM-containing composition significantly increased the amount of IGF-1 secretion. Therefore, it could be confirmed that the MSM-containing composition according to the present invention exhibits excellent effects of promoting secretion of IGF-1, which is an insulin-like growth factor

1. A pharmaceutical composition for preventing or treating obesity, comprising:
75 to 85% by weight (“wt. %”) of methylsulfonylmethane (MSM: dimethyl sulfone); 0.5 to 2 wt. % of vitamin B6 hydrochloride; 0.01 to 1 wt. % of folic acid; 2 to 8 wt. % of galactooligosaccharide; 10 to 18 wt. % of rice fermented magnesium; and 0.5 to 3 wt. % of vitamin C.

2. A pharmaceutical composition for preventing or treating fatty liver, comprising:
75 to 85 wt. % of methylsulfonylmethane (MSM: dimethyl sulfone); 0.5 to 2 wt. % of vitamin B6 hydrochloride; 0.01 to 1 wt. % of folic acid; 2 to 8 wt. % of galactooligosaccharide; 10 to 18 wt. % of rice fermented magnesium; and 0.5 to 3 wt. % of vitamin C.

3. A pharmaceutical composition for preventing or treating diabetes, comprising:
75 to 85 wt. % of methylsulfonylmethane (MSM: dimethyl sulfone); 0.5 to 2 wt. % of vitamin B6 hydrochloride; 0.01 to 1 wt. % of folic acid; 2 to 8 wt. % of galactooligosaccharide; 10 to 18 wt. % of rice fermented magnesium; and 0.5 to 3 wt. % of vitamin C.

4. A food composition for preventing or alleviating obesity, comprising:
75 to 85 wt. % of methylsulfonylmethane (MSM: dimethyl sulfone); 0.5 to 2 wt. % of vitamin B6 hydrochloride; 0.01 to 1 wt. % of folic acid; 2 to 8 wt. % of galactooligosaccharide; 10 to 18 wt. % of rice fermented magnesium; and 0.5 to 3 wt. % of vitamin C.

5. A food composition for preventing or improving fatty liver, comprising:
75 to 85 wt. % of methylsulfonylmethane (MSM: dimethyl sulfone); 0.5 to 2 wt. % of vitamin B6 hydrochloride; 0.01 to 1 wt. % of folic acid; 2 to 8 wt. % of galactooligosaccharide; 10 to 18 wt. % of rice fermented magnesium; and 0.5 to 3 wt. % of vitamin C.

6. A food composition for preventing or alleviating diabetes, comprising:
75 to 85 wt. % of methylsulfonylmethane (MSM: dimethyl sulfone); 0.5 to 2 wt. % of vitamin B6 hydrochloride; 0.01 to 1 wt. % of folic acid; 2 to 8 wt. % of galactooligosaccharide; 10 to 18 wt. % of rice fermented magnesium; and 0.5 to 3 wt. % of vitamin C.
